(19) United States
(12) Patent Application Publication
(10) Pub. No.: US 2013/0109638 A1
(43) Pub. Date: May 2, 2013

(54) ANTI-ACNE PHARMACEUTICAL COMPOSITION

(71) Applicant: Daniel Robert MILLER-GIBBONS, Mexico City (MX)

(72) Inventor: Daniel Robert MILLER-GIBBONS, Mexico City (MX)

(73) Assignee: FOLIA HEALTH PRODUCTS, S.A. DE C.V., entre Horacio y Mazarik (MX)

(21) Appl. No.: 13/286,377

(22) Filed: Nov. 1, 2011

(51) Int. Cl. A61K 31/704; A61P 17/10

(52) U.S. Cl. 514/26

(57) ABSTRACT

The invention relates to the field of the dermatologic therapy for acne; particularly it relates to a composition for treating acne, particularly nodular acne. The composition is made based on plant source proanthocyanidins and terpenoides.

### TECHNICAL FIELD

The invention relates to the field of the dermatologic therapy for acne; particularly, it relates to the field of treatment for nodular acne, and more particularly, to the treatment for sever nodular acne.

### BACKGROUND OF THE INVENTION

Acne is a problem of many teenagers and adults. The causes of acne are not completely known, although according to many researches the bacteria Propionibacterium acnes plays a significant role in some types of acne. This bacteria and other organisms naturally present may proliferate in the mixture of tallow and epithelial cells and promote inflammation.

It is believed that the origin of the disease is mainly due to a high tallow production, hypercornification of the infundibulum of the pilosebaceous glands, proliferation of the microbial flora (mainly Propionibacterium acnes) and a subsequent inflammation. The normal epidermal maturation process, named keratinization, involves the growing and expulsion of cells surrounding the pores and skin glands. In acne, this process is interrupted, causing an over production of epithelial cells (hyperkeratosis) in the follicular infundibulum of the sebaceous gland duct, forming a pore blocking or clogging.

The resulting injuries can be divided in inflammatory and non-inflammatory injuries. The non-inflammatory injuries, classified as open and closed comedos, are commonly known as black or white spots, respectively. The acne cases having only non-inflammatory injuries sometimes are referred to as mild acne.

Inflammatory injuries are a result of the excessive grow of the bacteria Propionibacterium acnes, and its interaction with the skin normal oils (tallow), resulting in the generation of biproducts causing an inflammatory reaction. Further to these primary injuries, patients may also suffer of scars as a complication of the inflammatory injuries. The acne inflammatory injuries may be divided in two groups. The least severe acne cases are associated with pustules and papules, as well as with non-inflammatory injuries. Papules are inflamed red moles without head ranging from 2 to 5 mm in diameter. The pustules are papules being at the surface and containing purulent gross material, thereby having a head with a white or yellow center. Depending on the number of papules or pustules present, the papule/pustule acne can be classified in a range from mild to severe. Individuals having severe cases of papule/pustule acne may have one or two acne nodules or cysts.

Many severe acne cases are associated with nodules or cysts as a predominant injury. Such individuals have three or more nodules and typically also have other non-inflammatory injuries, such as comedos. The cysts or nodules are obstructions of the skin sebaceous glands being open when exploding and produce inflammation and pus in the surrounding tissues. The nodules are large, up to 5 mm in diameter, and are below or inside of the skin surface, which can be painful and can last several months. The cysts are similar to nodules, but they are full of pus. The acne cases having an inflammatory condition with cysts and nodules often are referred to as severe acne. However, as there is not an accepted definition for the term “server acne” and often the papule or pustule acne are referred to as severe acne, it is preferably to make reference to the acne cases having cysts or nodules by the more specific term of nodular acne.

The Food and Drug Administration (FDA) has recognized that the nodular acne is a different entity which must be considered a different form from the acne mild forms.

The mild acne is typically treated with cleaners and benzoyl peroxide. The inflammatory mild acne is often treated with cleaners and keratolytic and comedolytic agents, such as retinoids (tretinoin, adapatene or tazarotene, salicylic acid, or alpha-hydroxy acids, often combined with topic or systemic antibiotics). The systemic antibiotics including tetracycline, minocycline, dioxycyclin, eritromicin, and azitromicin have been used to successfully treating the papule or pustule acne. Several of them have been approved by the FDA for such treatment. However, up to date any antibiotic has proved to be effective or has been approved by the FDA for the treatment of nodular acne. In some cases, the dermatologist recommends isotretinoin. However, the use of isotretinoin has severe inconveniences, since it has been proved that it causes issues in developing fetus, and then it is not recommended for pregnant women. In addition, the isotretinoin has been associated with depression events and suicide. In addition to the aforementioned, the isotretinoin has been associated with suicide and depression events. Due to the risks associated with the use of this antibiotic, the FDA has carried on a program to restrict its sale only by medical prescription.

In this manner, and due to the problems related to the nodular acne, there is the need for an effective therapy for the nodular acne avoiding severe side effects. Therefore, the present invention provides a pharmaceutical composition for treating acne.

### DESCRIPTION OF THE INVENTION

The present invention refers to a pharmaceutical composition for treating nodules associated with acne, such as in patients suffering from nodular acne, wherein the composition has as main ingredients proanthocyanidins of plant source and terpenoids.

As used herein, the plant source of proanthocyanidins is: the bark of the trees Mimosa tenuiflora, Pinus radiate, Acacia mangium, or Acacia mimosa.

As used herein, the terpenoids are: campesterol-3-O-beta-D-glucopiranosil, estigmasterol-3-O-beta-D-glucopiranosil, and beta-sitosterol-3-O-beta-D-glucopiranosil.

As used herein, the term “acne” means acne vulgaris.

As used herein, the term “nodule” in the context of the application is related to an acne injury that is a solid injury touchable higher than 5 mm in diameter and being deep inside the skin.

As used herein, the term “acne (or inflammatory acne) with presence of nodules” is referred to acne vulgaris wherein one or more nodules in the skin are present.

As used herein, the term “nodular acne” is related to the acne vulgaris wherein three or more nodules in the skin are present.

As used herein, the term “severe nodular acne” is referred to a case of acne vulgaris wherein the individual suffering of acne has five or more nodules present in the skin. Typically, patients with one or more acne nodules have additional manifestations of inflammatory acne, including multiple papules or pustule injuries.

The invention relates to a pharmaceutical composition which is applied topically over the acne nodules. In this manner, when applying it in an appropriate dose and time, the acne nodules are reduced in the patient's skin. This results in an enhancement in the physical appearance and the reduction or elimination of the disease and inconformity associated with the acne nodules.

The formulation of the invention can be found in any desired form, such as gel, cream, lotion, liquid or spraying mixture.

The composition can have other components typically present in a composition for the treatment of the skin, such as thickenings, emulsion stabilizers, emulsifiers, emollients, occlusive agents, skin conditioners, humectants, preservatives, antioxidants, pH adjusting agents, surfactants, quelating agents, fragances, etc. It is desirable that the composition has an aqueous base. Since some of the components are non-water soluble, the composition will need to form in an emulsion using emulsifying apparatuses available and well known in the state of the art, or using organic solvents miscible in water and added to dissolve the ingredients immiscible in water.

The thickeners include suitable polymers, such as carbomer, hydroxypropylmethyl cellulose, pVM/MA decadiene cross-linked polymer and acrylates/C₁₀₋₃₀ alkylacrylates cross-linked polymers in an amount generally of between 0.15 and 1.5%.

Another convenient ingredient is an emollient, such as diisopropyl adipate/isohexadecandimeticone and the C₁₂₋₁₅ alkylbenzoate, generally between 2 and 5%.

Among the skin conditioners some occlusive agents may be used, e.g., cyclomethicone, trimethylsiloxysylicate, glycerath26 or polyquaternium-7, generally in an amount between 1 and 4%.

The useful emulsifiers are, for example, cetyl alcohol, stearil, stearic acid, glyceril stearate, propylene glycol stearoyldioisostearoyl, lactilate, polyoxyethylen stearate.

Hydrating emulsions can be included, such as pantenol, generally in an amount between 0.25 and 1%.

Antioxidants may also be included, such as tocopheryl acetate or BTH, generally in an amount between 0.1 and 1%.

In addition, humectants may also be present, such as propylene glycol and glycerin, generally in an amount between 1 and 5%.

Suitable preservatives are present, such as phenoxyethanol and parabenes, generally in an amount between 0.5 and 1%.

A pH buffering agent typically will be a base, such as triethanolamine or sodium hydroxide in an amount enough to provide the desired pH.

A suitable fragrance can be added in an amount enough to give a pleasant smell.

The composition can also include a quelating agent, such as disodium EDTA or sodium citrate, in an amount between 0.01 and 0.1%.

The composition can also include sticking suppressing agents, such as aluminium starch-octenylsuccinate, in an amount between 1 and 2%.

The composition can be found in the liquid form with an aqueous base and a suitable organic solvent miscible with water to solubilize the lipophilic ingredients. A suitable solvent for this purpose is butylenglycol. It is also desirable to add a solubilizing agent such as polisorbate-20.

The following examples are shown with illustrative purposes only and shall not be considered in any way a limitative thereof. Any skill in the art could suggest multiple embodiments according with the state of the art. Said embodiments will form part of the present invention.

### Example 1

### Isolation of Proanthocyanidins

As a source of proanthocyanidins, the bark of the tree Mimosa tenuiflora was used. The bark was manually detached in the outer bark, which is of cork, and has a higher content of proanthocyanidins, and the inner bark, which is fibroses and has a lower content of antocyanidines. The detached outer bark was dried up to a humidity content of 20% or less, and it was further milled in a hammer miller to further pass through a mesh of 1.6 millimeters. The outer dust of the bark, pulverized to particle sizes of 1.6 millimeters or less was screened in eight fractions of the particle size in a shaker having seven meshes of 38, 45, 53, 63, 75, 90 and 106 mesh, respectively.

The eight fractions of the particle size obtained in the former step were subjected to a methanolic extraction for 15 minutes until obtaining proanthocyanidins/methanol solutions. The methanol was evaporated from the proanthocyanidins solution to obtain a concentrated solution, then, this concentrated solution of proanthocyanidins was subjected to freezing-drying to obtain the proanthocyanidins powder.

### Example 2

### Isolation of Propionibacterium acnes from Patients

After the clinical diagnosis of acne, the participants consented to have a biopsy of their injuries of inflamed acne using local anesthesia and a 4 mm needle. The biopsies were placed in a GAM culture broth (Nissui, Tokio, Japan), or in a Mueller-Hinton culture broth (Difco US), and were cultured at 37° C. for 18-24 h. The bacteria growing development was daily monitored.

### Example 3

### Determining the Growing Inhibition of Propionibacterium acnes with the Proanthocyanidins and Terpenoids Composition

After the culture and indentification of Propionibacterium acnes, the bacteria strains were adjusted to a 0.5 McFarland standard and used to inoculate GAM agar plates. Further, paper discs impregnated with the pharmaceutical composition at different concentrations of 0.5, 1.0, 1.5 and 2% of proanthocyanidins and terpenoids were located in agar plates. These agar plates were incubated in anaerobium ambient at 37° C. for 48 hours. Then, the inhibition zone was determined by measuring the diameter. The results show that all concentrations of proanthocyanidins and terpenoids inhibited the Propionibacterium acnes having the inhibition diameters of 15, 25 and 40 millimeters with respective concentrations of 0.5, 1.0, 1.5 and 2% of proanthocyanidins and terpenoids.

### Example 4

### Determining the Anti-Acne Activity in Vivo with the Proanthocyanidins and Terpenoids Composition

Teenagers ranging in 12-16 years old were selected with level 2 acne injuries in the back region. These injuries had the presence of comedos and pustule injuries with clear redness around the injuries but without cysts. Pictures were taken of the injuries before starting the treatment and every third day during the treatment. At the beginning of the treatment, the teenagers received instructions of how to apply topically the pharmaceutical gel (proanthocyanidins at 1.5%, terpenoids at 2%, polyehtylen glycol-200 at 5%, carbopol 940 at 0.5%, 1:1 methylprophylparaben at 0.5%, pH 7 adjusted with triethanolamine) once daily, after the shower, during 4 weeks. The gel is applied with the finger tips until the complete covering of the area with a fine layer, until the dark color of the preparation cannot be seen on the skin, it is not necessary to apply a thick layer. At the second day of the treatment, a considerable reduction of the inflammatory process and redness around the injuries could be seen. At the end of the treatment, 80% of the patients show a reduction in the number of pustule injuries. The constant use of the product prevented the occurring of new acne injuries. The preparation did not produce an irritating reaction over the skin or allergic reactions signs.

What is claimed is:

1. The use of proanthocyanidins and terpenoids to prepare a pharmaceutical composition useful for the treatment of nodular acne.

2. The use according to claim 1, wherein the proanthocyanidins are from a plant source.

3. The use according to claim 2, wherein the plant source is selected from the group of the bark of Mimosa tenuiflora, Pinus radiate, Acacia mangium, or Acacia mimosa.

4. The use according to claim 1, wherein the terpenoids are selected from the group of: campesterol-3-O-beta-D-glucopiranosil, estigmasterol-3-O-beta-D-glucopiranosil, and beta-sitosterol-3-O-beta-D-glucopiranosil.

5. A pharmaceutical composition for the treatment of nodular acne, wherein the composition comprises: a) proantocyanidenes from a plant source; b) terpenoids; and, c) a pharmaceutically acceptable carrier.

6. The composition according to claim 4, wherein the composition having a form selected from: gel, cream, lotion, liquid or a spray mixture.

7. The composition according to claim 4, wherein the pharmaceutically acceptable carrier is selected from the group of: thickenings, emulsion stabilizers, emulsifiers, emollients, occlusive agents, skin conditioners, humectants, humidifiers, preservatives, antioxidants, pH adjusting agents, surfactants, quelating agents, fragances.
